(19) United States
(12) Patent Application Publication
(10) Pub. No.: US 2010/0216876 A1
(43) Pub. Date: Aug. 26, 2010

(54) ANTIDIARRHETIC COMPOSITION, PRODUCT CONTAINING THE SAME AND METHOD OF PREVENTING DIAHHREA

(76) Inventors: Yuichi Suzuki, Shizuoka (JP); Kenya Ishida, Kanagawa (JP)

Correspondence Address: ABELMAN, FRAYNE & SCHWAB, 666 THIRD AVENUE, 10TH FLOOR, NEW YORK, NY, 10017, US

(21) Appl. No.: 12/681,245

(22) PCT Filed: Oct. 2, 2008
(86) PCT No.: PCT/JP2008/002769

§ 371 Date: Apr. 15, 2010

(30) Foreign Application Priority Data
Oct. 2, 2007 (JP) 2007-259140

(51) Int. Cl. A61K 31/045; C07C 35/17; C07C 43/196; C07C 49/11; C07C 233/01; A61K 31/075; A61K 31/215; A61K 31/12; A61K 31/16; A61P 1/12

(52) U.S. Cl. 514/529; 568/828; 568/670; 560/126; 568/376; 564/192; 514/729; 514/715; 514/690; 514/625

(57) ABSTRACT

An antidiarrhetic composition is provided which is efficacious against diarrhea, in particular, the predominant secretory diarrhea; an antidiarrhetic medicinal composition containing the same is also provided; a food containing the antidiarrhetic composition is also provided; and a method of preventing diarrhea using the same is also disclosed. The composition for relieving diarrhea is a cool composition capable of acting on the digestive tract in the recipient's body and inhibiting the secretion of the intestinal Cl⁻. In other words, a composition containing one or more kinds of cool act compounds. It may be provided by adding to medicines, foods or drinks.

### TECHNICAL FIELD

The present invention relates to an antidiarrhetic composition for relieving diarrhea, an antidiarrhetic medicinal composition containing the same, an antidiarrhetic composition-containing food, and a method of preventing diarrhea using the same.

### PRIOR ART

Diarrhea is a problem common to people of all ages and animals in general such as livestock. Diarrhea may occur in adults because of stress. For infants, it may cause serious dehydration, thereby threatening the maintenance of life.

Generally, the amount of feces in a healthy adult is about 150 g/day, and the amount of fluid in the feces is concentrated to about 100 to 150 ml/day. The indication of diarrhea is such that the daily amount of fluid in the feces is 200 ml or more, or the daily weight of the feces is 200 g or more. The approximate amounts of fluids flowing into the small intestine per day are as follows: orally-ingested fluid: 2 L, saliva: 1 L, gastric fluid: 2 L, pancreatic fluid: 2 L, and bile: 1 L; additionally, 1 L of intestinal fluid is secreted. Since the amount of fluid passing through the ileocecal junction is 1.5 to 2 L, 7 to 8 L of fluid is absorbed by the small intestine, and about 1.5 L of fluid is further absorbed by the large intestine.

Diarrhea is classified into six types on basis of the mechanisms of development: osmotic diarrhea, exudative diarrhea, secretory diarrhea, diarrhea caused by abnormal intestinal motility, diarrhea caused by abnormal active ion transport, and pathophysiologically unknown other types of diarrhea. These mechanisms hardly work independently, and some of them are mostly combined to develop diarrhea.

Osmotic diarrhea occurs because a large amount of poorly absorbable and hyperosmotic solute is present in the intestinal tract, and thereby fluids are moved to the intestinal lumen. This type of diarrhea is associated with food ingestion and can be inhibited by fasting. Exudative diarrhea occurs because intestinal inflammation leads to the increased permeability of the intestinal tract wall, and a large amount of exudate is excreted into the lumen. Blood, pus, and mucus are often adhered to the feces. Although this type of diarrhea worsens with eating, it is not completely cured even by fasting. Secretory diarrhea is caused by abnormally increased secretion from the mucosa of the digestive tract. There are two mechanisms: cyclic AMP (antiallergic substance)-meditated or non-meditated mechanisms. Various gastrointestinal hormones, enterotoxins, etc. are involved in these mechanisms. This type of diarrhea features a huge amount of watery diarrhea and cannot be cured by fasting. Diarrhea caused by abnormal intestinal motility may be resulted from increased or decreased intestinal motility. When diarrhea occurs because of decreased intestinal motility, delayed passage of small intestine contents results in bacterial growth in the small intestine and causes the deconjugation of bile acids. As a result, the absorption of fat or water is impaired, thereby producing diarrhea. Diarrhea caused by abnormal active ion transport is resulted from a congenital defect in absorption of Cl⁻ in the ileum. This type of diarrhea, which is a rare disease that may be found in infants, can be cured by fasting. Pathophysiologically unknown other types of diarrhea have been found in Addison disease, hypoparathyroidism, liver cirrhosis, Mg deficiency, and the like.

The primary cause of secretory diarrhea, which is a predominant type of diarrhea, is believed to be the abnormal activation of intestinal Cl⁻ secretion.

As an effective preventive measure against this type of diarrhea, the present inventors found that menthol and menthone function as active ingredients, and disclosed this finding in Patent Document 1.

### Patent Document 1: Japanese Patent Application No. 2006-097890

However, the potency of menthol and menthone was not always satisfactory. In order to obtain the desired effect, it was necessary to ingest large amounts of these compounds.

Menthol and menthone are known to provide a cooling sensation. However, there has been no finding as to whether such a cooling sensation has a therapeutic effect on diarrhea.

### SUMMARY OF INVENTION

### Technical Problem

Accordingly, an object of the present invention is to provide an antidiarrhetic composition that is particularly effective in relieving secretory diarrhea, which is a predominant type of diarrhea, using a menthol derivative having various cooling functions; an antidiarrhetic medicinal composition containing the same; an antidiarrhetic composition-containing food; and a method of preventing diarrhea using the same.

### Solution to Problem

In order to achieve the above object, the antidiarrhetic composition of the present invention has the following features. That is, the antidiarrhetic composition for relieving diarrhea is characterized by containing one or more kinds of cooling compositions, i.e., compounds that provide a cooling sensation, the cooling composition being capable of acting on the digestive tract of an organism that has ingested the composition, to inhibit intestinal Cl⁻ secretion.

Here, the cooling composition is preferably any one of isopulegol, 3-(menthoxy)propane-1,2-diol, 2-(menthoxy)ethanol, 2-[2-(menthoxy)ethoxy]ethanol, 3-(menthoxy)propanol, 2-methyl-3-(menthoxy)propane-1,2-diol, p-menthane-3,8-diol, menthyl 3-hydroxybutanoate, 1-(2-hydroxy-4-methyl-cyclohexyl)-ethanone, N-ethyl-menthyl carboxamide, menthyl lactate, and N-methyl-2,2-isopropylmethyl-3-methylbutanamide. Optically active substances thereof are more preferred, and l-forms thereof are even more preferred.

Further, the cooling composition preferably has a p-menthane skeleton and has a polar site at the 3-position of the skeleton.

The cooling composition may be used in the form of a mixture with menthol.

The antidiarrhetic composition may be provided in the form of a mixture with a medicine, food, or drink. More specifically, the antidiarrhetic medicinal composition of the present invention, which is a medicine containing a composition for relieving diarrhea, is characterized by containing the above antidiarrhetic composition as a main ingredient of the medicine, together with other compositions in a unified form.

Similarly, the antidiarrhetic composition-containing food of the present invention, which is a food or drink containing a composition for relieving diarrhea, is characterized in that it is prepared by incorporating the above antidiarrhetic composition in other food or drink.

The antidiarrhetic composition etc. described above may be used in a method of preventing diarrhea of humans and animals such as livestock. More specifically, the method of the present invention for preventing diarrhea is characterized by previously administering the antidiarrhetic composition, antidiarrhetic medicinal composition, or antidiarrhetic composition-containing food to a subject in a significant amount sufficient to act on the digestive tract of the organism to inhibit intestinal Cl secretion, thereby preventing diarrhea.

### EFFECT OF INVENTION

The antidiarrhetic composition, antidiarrhetic medicinal composition containing the same, and antidiarrhetic composition-containing food of the present invention effectively contribute to the relief of diarrhea, such as secretory diarrhea, without side effects, because of the inhibitory action of the cooling composition on intestinal Cl⁻ secretion. They also contribute to the prevention of diarrhea by the previous ingestion of predetermined amounts of these compositions.

### DESCRIPTION OF EMBODIMENTS

The following describes embodiments of the present invention with reference to drawings. The embodiments can be suitably modified without departing from the scope of the invention. The present inventor focused on the group of compounds having a cooling function as an antidiarrhetic composition, and the validity of the compounds was examined for verification.

Some cooling agents including menthol derivatives are known to have a mosquito repellent effect; however, most of their physiological activity remains unknown, and there have been no reports regarding the inhibitory effect of the cooling agents on intestinal Cl⁻ secretion. If cooling compositions are confirmed to have an inhibitory effect on Cl⁻ secretion, they are expected to have an antidiarrheal effect. For this reason, whether cooling compositions could inhibit intestinal Cl⁻ secretion was examined.

A sample of large-intestinal mucosa was prepared in the following manner. A mouse was killed by cervical vertebra dislocation. After abdominal incision, the cecum was excised by cutting the boundary parts of the cecum with the small intestine and the large intestine. The cecum was cut open with scissors into a sheet. In order to completely remove the contents of the cecum, the cecum was pinched with tweezers and washed with a buffered solution. Then, a substitution fluid was placed in a petri dish covered with rubber, and the cecum was attached thereon with the serosa side up. Under conditions in which the buffered solution-in the petri dish was always aerated with 95% O₂/5% CO₂, the muscle layer was peeled using tweezers, and a sample composed of mucosa and submucosa was produced. The sample was divided into four pieces for the experiment.

Electrical analysis was carried out using this sample. FIG. 1 is a diagram showing an embodiment of measurement of short-circuit current (Isc) in Ussing chamber. The mucosa sample was placed between two Ussing-type chambers (window area: 0.2 cm²) containing 5 ml of buffered solution. For the measurement of the electrical potential difference, a pair of calomel electrodes was connected to each chamber through a 1MKCl/2% agar salt bridge. For passing current, Ag/AgGl electrodes connected through a 1MNaCl/2% agar salt bridge were mounted. These electrodes were connected to a voltage clamp apparatus, and the short-circuit current (Isc) was measured. As for Isc, the current flowing from the mucosa side to the serosa side was positive.

The solutions to be administered were prepared as stock solutions so that the concentration of each solution was 1,000 times higher than the desired final concentration. More specifically, forskolin (FK) was prepared at a concentration of 5 mM in DMSO; tetrodotoxin was prepared at a concentration of 300 μM in distilled water; cooling compositions were prepared in DMSO at concentrations of 1,000 mM, 500 mM, 150 mM, 50 mM, and 20 mM; and bumetanide was prepared at a concentration of 50 mM in DMSO.

FIG. 2 is a graph showing the effect of menthol administered on the serosa side after the administration of forskolin (FK), on Isc compared with vehicle administration. When forskolin, which increases intracellular cAMP levels, was administered on the serosa side, Isc significantly increased. At least part of such a cAMP-dependent increase in Isc is attributable to the activation of Cl⁻ secretion mechanism. Subsequently, when 500 μM of menthol was administered on the serosa side, Isc significantly decreased. The decreased value of Isc was 72.07±6.78 μM.

The antidiarrhetic composition, antidiarrhetic medicinal composition containing the same, or antidiarrhetic composition-containing food of the present invention may contain additional various medicinal components, if necessary, or may be used in combination. The type and total amount of such medicinal components are not limited. For example, antacids, stomachics, digestives, antiflatulents, other antidiarrheals, analgesic and antispasmodic agents, vitamins, amino acids, and other herbal medicines can be used. Specific examples of additional components that can suitably be used in the present invention are shown below.

Examples of antacids include magnesium-based antacids, such as dried aluminum hydroxide gel, magnesium aluminosilicate, magnesium aluminometasilicate, aluminum silicate, hydrotalcite, magnesia alumina hydrate, aluminum hydroxide gel, coprecipitation product of aluminum hydroxide and sodium hydrogencarbonate, aluminum hydroxide-magnesium carbonate co-dried gel, coprecipitation product of aluminum hydroxide, calcium carbonate, and magnesium carbonate, magnesium carbonate, magnesium oxide, magnesium hydroxide, magnesium silicate, and coprecipitation product of magnesium hydroxide and potassium aluminum sulfate; calcium-based antacids, such as anhydrous calcium hydrogen phosphate, calcium hydrogen phosphate, precipitated calcium carbonate, calcium lactate, and calcium hydroxide; sodium-based antacids, such as sodium hydrogen carbonate, sodium citrate, and sodium acetate; anionic exchange resins, such as polyamino methylene resins; H2-receptor antagonists, such as famotidine, ranitidine, and cimetidine; proton pump inhibitor; additionally, gastric mucin, squid bone, Haliotis diversicolor, oyster, aminoacetic acid, dihydroxyaluminum aminoacetate, scopolia extract, and the like.

Examples of stomachics include herbal medicines, such as aniseed, aloe, fennel, turmeric, linderae radix, Rabdosia japonica, scutellaria root, phellodendron bark, coptis rhizome, processed garlic, zedoary, pogostemon herb, cinchona bark, nux vomica, Zingiber officinale, calamus root, dried ginger, trifoliate orange, immature orange, cinnamon bark, gentian, red ginseng, magnolia bark, evodia fruit, pepper, calumba, condurango, zanthoxylum fruit, Hedychium spicatum, perilla seed, amomum seed, ginger, cardamon, Citrus reticulata, sweet-flag root, Centaurium minus, swertia herb, atractylodes lancea rhizome, perilla herb, star anise, rhubarb, Panax japonicus rhizome, clove, citrus unshiu peel, capsicum, bitter orange peel, animal bile, picrasma wood, nutmeg, ginseng, mentha herb, Piper longum, atractylodes rhizome, hop, nux vomica extract, Menyanthes trifoliata, saussurea root, bitter cardamon, Japanese gentian, Alpinia officinarum rhizome, sophora root, Rhus javanica, crataegus fruit, Myrica rubra, mallotus bark, gambir, Prunus mume, cassia seed, and geranium herb; parasympathomimetic agents, such as carnitine, neostigmine, bethanechol, carpronium, and tolazoline; antidopaminergic drugs, such as metoclopramide, domperidone, and sulpiride; trimebutine, glutamic acid, and the like.

Examples of digestives include starch-digesting enzyme, protein-digesting enzyme, fat-digesting enzyme, cellulose-digesting enzyme, ursodeoxycholic acid, oxycholanic acid hydrochloride, cholic acid, bile powder, bile extract, dehydrocholic acid, animal bile, and the like. Examples of the above-described enzymes include diastase, pancreatin, pepsin, ptyalin, β-galactosidase, amylase, trypsin, papain, protease, lipase, cellulase, pancreatin, and the like.

Examples of antiflatulents include components of bacteria that regulate intestinal functions, gambir, Prunus mume, cassia seed, geranium herb, and the like.

Examples of other antidiarrheals include acrinol, berberine chloride, guaiacol, creosote, phenyl salicylate, guaiacol carbonate, berberine tannate, bismuth subsalicylate, bismuth subnitrate, bismuth subcarbonate, bismuth subgallate, tannic acid, albumin tannate, methylenethymoltannin, kaolin, natural aluminum silicate, aluminum hydroxynaphthoate, pectin, medicinal carbon, precipitated calcium carbonate, calcium lactate, calcium hydrogen phosphate, gambir, Prunus mume, phellodendron bark, coptis rhizome, sophora root, geranium herb, Rhus javanica, crataegus fruit, swertia herb, Myrica rubra, and the like.

Examples of analgesic and antispasmodic agents include papaverine hydrochloride, ethyl aminobenzoate, scopolamine hydrobromate, scopolamine methylbromide, corydalis tuber, glycyrrhiza, magnolia bark, peony root, timepidium bromide, oxyphencyclimine hydrochloride, dicyclomine hydrochloride, methixene hydrochloride, atropine methylbromide, 1-hyoscyamine methylbromide, methylbenactyzium bromide, belladonna extract, scopolia extract, diphenylpiperidinomethyldioxolan iodide, total alkaloid citrate of scopolia rhizome, and the like.

Specific examples of vitamins are as follows. Examples of vitamin A include retinal, retinol, retinoic acid, carotene, dehydroretinol, lycopene, pharmaceutically acceptable salts thereof (e.g., retinol acetate, retinol palmitate, etc.), and the like. Examples of vitamin B include thiamine, thiamine disulfide, dicethiamine, octotiamine, cyclotiamine, bisibutiamine, bisbentiamine, prosultiamine, benfotiamine, fursultiamine, riboflavin, flavin adenine dinucleotide, pyridoxine, pyridoxal, hydroxocobalamin, cyanocobalamin, methylcobalamin, deoxy adenovirus cobalamine, folic acid, tetrahydrofolic acid, dihydrofolic acid, nicotinic acid, nicotinamide, nicotinyl alcohol, pantothenic acid, panthenol, biotin, choline, inositol or pharmaceutically acceptable salts thereof (e.g., thiamin hydrochloride, thiamine nitrate, dicethiamine hydrochloride, fursultiamine hydrochloride, riboflavin butyrate, riboflavin sodium phosphate, flavin-adenine dinucleotide sodium, pyridoxine hydrochloride, pyridoxal phosphate, pyridoxal calcium phosphate, hydroxocobalamin hydrochloride, hydroxocobalamin acetate, calcium pantothenate, sodium pantothenate, etc.), and the like. Examples of vitamin C include ascorbic acid, erythorbic acid, derivatives or pharmaceutically acceptable salts thereof (e.g., sodium ascorbate, sodium erythorbate, etc.), and the like. Examples of vitamin D include ergocalciferol, cholecalciferol, hydroxycholecalciferol, dihydroxycholecalciferol, dihydrotachysterol, pharmaceutically acceptable salts thereof, and the like. Examples of vitamin E include tocopherol and derivatives thereof, ubiquinone derivatives and pharmaceutically acceptable salts thereof (tocopherol acetate, tocopherol nicotinate, tocopherol succinate, tocopherol calcium succinate, etc.), and the like. Examples other vitamins include hesperidin, carnitine, ferulic acid, γ-orizanol, orotic acid, rutin, eriocitrin, pharmaceutically acceptable salts thereof (carnitine chloride etc.), and the like.

Examples of amino acids include leucine, isoleucine, valine, methionine, threonine, alanine, phenylalanine, tryptophan, lysine, asparagine, aspartic acid, serine, glutamine, glutamic acid, proline, tyrosine, cysteine, histidine, ornithine, hydroxyproline, hydroxylysine, aminoethylsulfonic acid, pharmaceutically acceptable salts thereof (an equal proportion mixture of potassium aspartate and magnesium aspartate, cysteine hydrochloride, etc.), and the like.

Examples of herbal medicines include processed garlic, ginseng, coix seed, camomile, cinnamon bark, kakkonto, ephedra herb, Nandina domestica, Prunus jamasakura, polygala root, glycyrrhiza, apricot kernel, plantago seed, plantago herb, Lycoris radiata, senega, ipecac, fritillaria bulb, gambir, fennel, scutellaria root, trichosanthes seed, oriental bezoar, schisandra fruit, asiasarum root, Aster tataricus, musk, Glehnia littoralis, ginger, mulberry bark, perilla herb, Panax japonicus rhizome, citrus unshiu peel, ophiopogon tuber, pinellia tuber, and the like.

The amount of the above additional components added may be varied depending on various factors including the desired effect and the age and condition of a subject. For example, the amount thereof may be 0.001 to 80 mass %, preferably 0.001 to 30 mass %, and more preferably 0.001 to 10 mass %, based on the total amount of the antidiarrhetic composition, antidiarrhetic medicinal composition, or antidiarrhetic composition-containing food.

The dosage form of the antidiarrhetic composition, antidiarrhetic medicinal composition, or antidiarrhetic composition-containing food of the present invention is not limited, and any dosage forms that are generally used are available. The antidiarrhetic composition, antidiarrhetic medicinal composition, or antidiarrhetic composition-containing food of the present invention is generally in the form of a solid, semi-solid, or liquid formulation; solid or liquid formulations (e.g., decoctions, infusions, etc.) are preferred, and solid formulations are most preferred. For example, the formulations of the present invention may be in the form of tablets (including uncoated tablets, sugar-coated tablets, intraorally fast-disintegrating tablets, intraorally fast-dissolving tablets, chewable tablets, effervescent tablets, lozenges, drops, film-coated tablets, etc.), pills, granules, subtle granules, powders, hard capsules, and soft capsules, more preferably tablets, and particularly preferably dosage forms including intraorally fast-disintegrating tablets, intraorally fast-dissolving tablets, chewable tablets, etc., which can easily be taken without water when symptoms of diarrhea appear, or dosage forms including sugar-coated tablets, film-coated tablets, etc., which can block unpleasant tastes.

In addition to the above-described components, the antidiarrhetic composition, antidiarrhetic medicinal composition, or antidiarrhetic composition-containing food of the present invention may suitably contain any components that can generally be used in drugs, quasi drugs, and food products, depending on the application, dosage form, etc., as long as the effect of the present invention, pharmaceutical stability, etc., are not impaired. For example, such components may be carrier components or additives, although not limited thereto. Examples of carrier components or additives that can be added to the solid formulation include excipients, disintegrants, binders, lubricants, antioxidants, coating agents, coloring agents, flavoring substances, surfactants, plasticizers, sweetening agents, flavoring agents, disintegration aids, foaming agents, adsorbents, preservatives, wetting agents, antistatic agents, and the like. Examples of carrier components or additives that can be added to the liquid formulation include solvents, pH adjusters, refreshing agents, suspending agents, defoaming agents, thickening agents, solubilizing agents; and surfactants, antioxidants, coloring agents, sweetening agents, and flavoring agents, as described above; additionally, antiseptic and antibacterial agents, chelating agents, solubilizers or solubilizing agents, stabilizers, fluidizers, emulsifiers, thickeners, buffers, isotonizing agents, dispersants, and the like. Specific examples of such usable components are shown below, although not limited thereto.

Examples of excipients include sugar alcohols such as D-sorbitol, mannitol, and xylitol; saccharides such as glucose, sucrose, lactose, and fructose; crystalline cellulose, carmellose sodium, croscarmellose sodium, calcium hydrogen phosphate, wheat starch, rice starch, corn starch, potato starch, dextrin, β-cyclodextrin, light anhydrous silicic acid, titanium oxide, magnesium aluminometasilicate, talc, kaolin, and the like. Preferred excipients are mannitol, croscarmellose sodium, and light anhydrous silicic acid, although not limited thereto.

Examples of disintegrants include low-substituted hydroxypropyl cellulose, calcium carboxymethyl cellulose, croscarmellose sodium, hydroxypropyl starch, partially pregelatinized starch, and the like.

Examples of binders include methylcellulose, ethylcellulose, hydroxypropyl cellulose, hydroxypropyl methylcellulose, and other cellulose derivatives, polyvinyl pyrrolidone, polyvinyl alcohol, acrylic acid-based polymer, gelatin, gum arabic, pullulan, pregelatinized starch, agar, tragacanth, sodium alginate, propylene glycol alginate, and the like.

Examples of lubricants include stearic acid, magnesium stearate, calcium stearate, polyoxyl stearate, cetanol, talc, hardened oil, sucrose fatty acid ester, dimethylpolysiloxane, beeswax, white beeswax, and the like. A preferred lubricant is magnesium stearate, although not limited thereto.

Examples of antioxidants include dibutylhydroxytoluene (BHT), propyl gallate, butylhydroxyanisole (BHA), tocopherol, citric acid, and the like.

Examples of coating agents include hydroxypropyl methylcellulose, hydroxypropyl cellulose, methylcellulose, ethylcellulose, hydroxypropyl methylcellulose phthalate, hydroxypropyl methylcellulose acetate succinate, carboxymethyl ethyl cellulose, cellulose acetate phthalate, polyvinyl acetal diethylaminoacetate, aminoalkylmethacrylate copolymer, hydroxypropyl methylcellulose acetate succinate, methacrylic acid copolymer, polyvinyl acetate diethylaminoacetate, shellac, and the like.

Examples of coloring agents include food red No. 2, food red No. 3, food red No. 102, food yellow No. 4, food yellow No. 5, food blue No. 1, food yellow No. 4 metal lake, sodium copper chlorophyllin, riboflavin, turmeric extract, carotene liquid, and the like.

Examples of flavoring substances include aspartame, ascorbic acid, stevia, menthol, crude glycyrrhiza extract, simple syrup, and the like.

Examples of surfactants include polyoxyethylene hydrogenated castor oil, glyceryl monostearate, sorbitan monostearate, sorbitan monolaurate, polyoxyethylene polyoxypropylene, polysorbates, sodium lauryl sulfate, macrogols, sucrose fatty acid ester, and the like.

Examples of plasticizers include triethyl citrate, polyethylene glycol, triacetin, cetanol, and the like.

Examples of sweetening agents include natural or synthetic sweetening agents, such as sucrose, mannitol, and aspartame.

Examples of flavoring agents include camphor, borneol, cinnamaldehyde, and the like.

Examples of solvents include water, ethanol, isopropanol, lauryl alcohol, cetanol, stearyl alcohol, oleyl alcohol, lanolin alcohol, behenyl alcohol, 2-hexyl decanol, isostearyl alcohol, 2-octyl dodecanol, and the like.

Examples of pH adjusters include citric acid, malic acid, sodium hydrogen phosphate, dipotassium phosphate, and the like.

Examples of suspending agents include kaolin, carmellose sodium, xanthan gum, methylcellulose, tragacanth, and the like.

Examples of defoaming agents include dimethylpolysiloxane, silicon defoaming agent, and the like.

Examples of thickening agents include xanthan gum, tragacanth, methylcellulose, dextrin, and the like.

Examples of solubilizing agents include ethanol, sucrose fatty acid ester, macrogol, and the like.

The antidiarrhetic composition, antidiarrhetic medicinal composition, or antidiarrhetic composition-containing food of the present invention can be produced by a method generally used in the technical field without modification or with suitable modification. For example, tablets can be prepared by mixing a powdered active ingredient and a pharmaceutically acceptable carrier component (e.g., an excipient), and directly compression-molding the mixture (direct tableting method). Drops may be prepared by pouring the mixture into a mold. Among the solid formulations, powders such as granules may be prepared by various granulation methods (extrusion granulation method, crushing granulation method, dry compression granulation method, fluidized-bed granulation method, rolling granulation method, high-speed agitated granulation method, etc.). Tablets can also be prepared by suitably combining such a granulation method, a tableting method (wet tableting method etc.) and the like (indirect tableting method). Further, capsules can be prepared by filling capsules (soft or hard capsules) with powder formulations (dust formulations, granules, etc.) using a conventional method. The tablets may be coated with sugar or film to prepare sugar- or film-coated tablets. Moreover, the tablets may be in the form of single-layer tablets or laminated tablets such as double-layer tablets. The liquid formulations can be prepared by dissolving or dispersing each component in an aqueous medium (purified water, heat-purified water, ethanol-containing purified water, or the like), which is a carrier component, optionally followed by heating, filtration, fabric filtration or sterilization, and placing the resultant mixture in a predetermined container, followed by sterilization etc.

### Example 1

FIG. 3 is a graph showing the effect of 2-methyl-3-(l-menthoxy)propane-1,2-diol administered on the serosa side after the administration of forskolin, on Isc. When forskolin, which increases intracellular cAMP levels, was administered on the serosa side, Isc significantly increased. At least part of such a cAMP-dependent increase in Isc is attributable to the activation of Cl⁻ secretion mechanism. Subsequently, when 20 μM of 2-methyl-3-(l-menthoxy)propane-1,2-diol was administered on the serosa side, Isc decreased by about 100 μA/cm². This action is almost equal to the administration of 500 μM of menthol. The dose dependence was observed at doses up to 50 μM, although it was not saturated at dosages of 150 μM or more.

### Example 2

Under the same conditions as in Example 1, the effect of 3-(l-menthoxy)propanol administered on the serosa side after the administration of forskolin, on Isc was measured. As a result of the administration of 3-(l-menthoxy)propanol on the serosa side, Isc decreased by about 50 μA/cm² at a dose of 20 μM, and about 70 μA/cm² at 50 μM.

### Example 3

Under the same conditions as in Example 1, the effect of 3-(l-menthoxy)propane-1,2-diol administered on the serosa side after the administration of forskolin, on Isc was measured. As a result of the administration of 3-(l-menthoxy)propane-1,2-diol on the serosa side, Isc decreased by about 50 μA/cm² at a dose of 20 μM, and about 70 μA/cm² at 50 μM.

### Example 4

Under the same conditions as in Example 1, the effect of 2-(l-menthoxy)ethanol administered on the serosa side after the administration of forskolin, on Isc was measured. As a result of the administration of 2-(l-menthoxy)ethanol on the serosa side, Isc decreased by about 10 μA/cm² at a dose of 20 μM, and about 70 μA/cm² at 150 μM.

In addition to the cooling compositions used in the above examples, the same effect will be obtained by using l-isopulegol, 2-[2-(l-menthoxy)ethoxy]ethanol, p-menthane-3,8-diol, l-menthyl 3-hydroxybutanoate, 1-(2-hydroxy-4-methyl-cyclohexyl)-ethanone, N-ethyl-l-menthyl carboxamide, l-menthyl lactate, N-methyl-2,2-isopropylmethyl-3-methylbutanamide, or cooling compositions equivalent thereto.

Moreover, although the experimental results of the above examples have been obtained by using L-forms, the similar effect was obtained by using optical isomers such as D- and DL-forms.

### INDUSTRIAL APPLICABILITY

As described above, the present invention has demonstrated that cooling compositions can inhibit intestinal Cl⁻ secretion activated by cAMP. Since secretory diarrhea, which causes the majority of diarrhea, is presumably caused by abnormal activation of intestinal Cl⁻ secretion, the foods and drinks of the present invention, which contain a predetermined significant amount of such a cooling composition, are effective in relieving and preventing diarrhea and are industrially very useful since they can be easily ingested without side effects.

### BRIEF DESCRIPTION OF DRAWINGS

FIG. 1 is a diagram showing an embodiment of measurement of short-circuit current (Isc) in Ussing chamber.

FIG. 2 is a graph showing the effect of menthol administered on the serosa side after the administration of forskolin, on Isc compared with vehicle administration.

FIG. 3 is a graph showing the dose dependence of the Isc inhibitory effect of 2-methyl-3-(l-menthoxy)propane-1,2-diol administered on the serosa side after the administration of forskolin a typical trace.

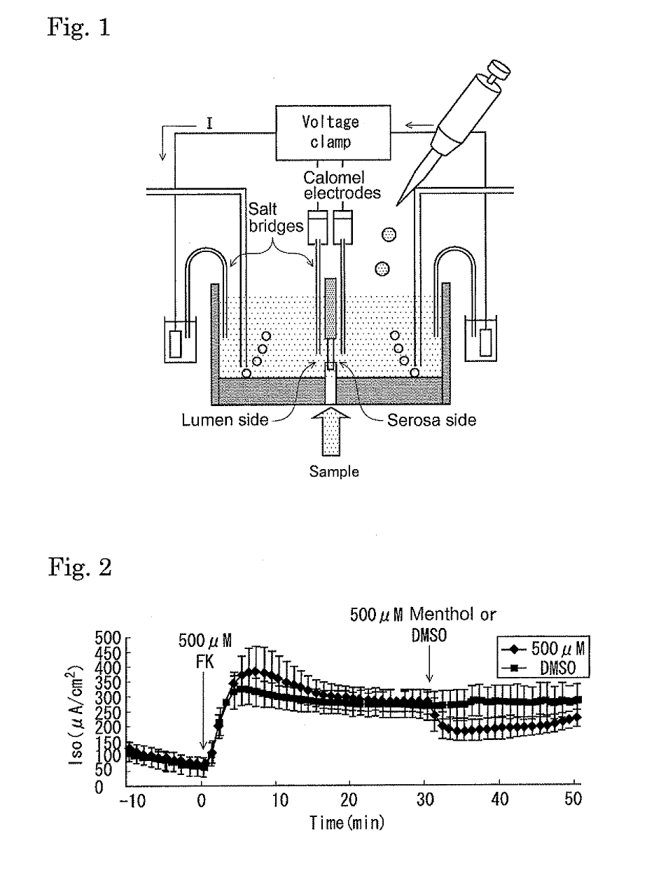

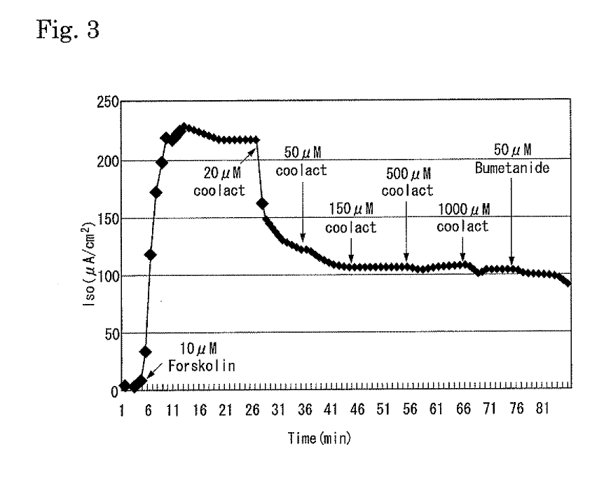

1. An antidiarrhetic composition for relieving diarrhea, comprising one or more kinds of cooling compositions that are capable of acting on the digestive tract of a subject that has ingested the composition, to inhibit intestinal Cl⁻ secretion.

2. The antidiarrhetic composition according to claim 1, wherein the cooling composition is any one of isopulegol, 3-(menthoxy)propane-1,2-diol, 2-(menthoxy)ethanol, 2-[2-(menthoxy)ethoxy]ethanol, 3-(menthoxy)propanol, 2-methyl-3-(menthoxy)propane-1,2-diol, p-menthane-3,8-diol, menthyl 3-hydroxybutanoate, 1-(2-hydroxy-4-methyl-cyclohexyl)-ethanone, N-ethyl-menthyl carboxamide, menthyl lactate, and N-methyl-2,2-isopropylmethyl-3-methylbutanamide.

3. The antidiarrhetic composition according to claim 1, wherein the cooling composition has a p-menthane skeleton and has a polar site at the 3-position of the skeleton.

4. The antidiarrhetic composition according to claim 1, wherein the cooling composition is a mixture with menthol.

5. An antidiarrhetic medicinal composition, which is a medicine containing a composition for relieving diarrhea, the medicinal composition comprising the antidiarrhetic composition according to claim 1 as a main ingredient of the medicine, together with other compositions in a unified form.

6. An antidiarrhetic composition-containing food, which is a food or drink containing a composition for relieving diarrhea, the food being prepared by incorporating the antidiarrhetic composition according to claim 1 in other food or drink.

7. A method of preventing diarrhea by previously administering to a subject the antidiarrhetic composition according to claim 1 in a significant amount sufficient to act on the digestive tract of the subject to inhibit intestinal Cl⁻ secretion.

8. The antidiarrhetic composition according to claim 2, wherein the cooling composition has a p-menthane skeleton and has a polar site at the 3-position of the skeleton.

9. The antidiarrhetic composition according to claim 2, wherein the cooling composition is a mixture with menthol.

10. The antidiarrhetic composition according to claim 3, wherein the cooling composition is a mixture with menthol.

11. An antidiarrhetic medicinal composition, which is a medicine containing a composition for relieving diarrhea, the medicinal composition comprising the antidiarrhetic composition according to claim 2 as a main ingredient of the medicine, together with other compositions in a unified form.

12. An antidiarrhetic medicinal composition, which is a medicine containing a composition for relieving diarrhea, the medicinal composition comprising the antidiarrhetic composition according to claim 3 as a main ingredient of the medicine, together with other compositions in a unified form.

13. An antidiarrhetic medicinal composition, which is a medicine containing a composition for relieving diarrhea, the medicinal composition comprising the antidiarrhetic composition according to claim 4 as a main ingredient of the medicine, together with other compositions in a unified form.

14. An antidiarrhetic composition-containing food, which is a food or drink containing a composition for relieving diarrhea, the food being prepared by incorporating the antidiarrhetic composition according to claim 2 in other food or drink.

15. An antidiarrhetic composition-containing food, which is a food or drink containing a composition for relieving diarrhea, the food being prepared by incorporating the antidiarrhetic composition according to claim 3 in other food or drink.

16. An antidiarrhetic composition-containing food, which is a food or drink containing a composition for relieving diarrhea, the food being prepared by incorporating the antidiarrhetic composition according to claim 4 in other food or drink.

17. A method of preventing diarrhea by previously administering to a subject the antidiarrhetic composition according to claim 2 in a significant amount sufficient to act on the digestive tract of the subject to inhibit intestinal Cl⁻ secretion.

18. A method of preventing diarrhea by previously administering to a subject the antidiarrhetic composition according to claim 3 in a significant amount sufficient to act on the digestive tract of the subject to inhibit intestinal Cl⁻ secretion.

19. A method of preventing diarrhea by previously administering to a subject the antidiarrhetic composition according to claim 4 in a significant amount sufficient to act on the digestive tract of the subject to inhibit intestinal Cl⁻ secretion.

20. A method of preventing diarrhea by previously administering to a subject the antidiarrhetic medicinal composition according to claim 5 in a significant amount sufficient to act on the digestive tract of the subject to inhibit intestinal Cl⁻ secretion.

21. A method of preventing diarrhea by previously administering to a subject the antidiarrhetic composition-containing food according to claim 6 in a significant amount sufficient to act on the digestive tract of the subject to inhibit intestinal Cl⁻ secretion.
